(19) United States
(12) Patent Application Publication
(10) Pub. No.: US 2022/0348982 A1
(43) Pub. Date: Nov. 3, 2022

(54) USING BIO-MARKERS FOR OIL EXPLORATION

(71) Applicant: Saudi Arabian Oil Company, Dhahran (SA)

(72) Inventors: Abdulmohsen A. Al-Humam, Dammam (SA); Mahdi A. AbuAli, Dhahran (SA); Manar M. AlAhmari, Dhahran (SA); Mohammed A. Bataweel, Dhahran (SA)

(21) Appl. No.: 17/244,283

(22) Filed: Apr. 29, 2021

(51) Int. Cl. C12Q 1/64; G16B 40/30; C12Q 1/6888; C12Q 1/686

(52) U.S. Cl. CPC C12Q 1/64; G16B 40/30; C12Q 1/6888; C12Q 1/686; C12Q 2600/156; C12Q 2600/158

(57) ABSTRACT

A method for using genomic data to locate a reservoir is provided. The method includes collecting samples in a field over a reservoir. A genomic analysis is performed on the samples to obtain genomic data. The genomic data is clustered to classify sequences of microbial communities associated with using hydrocarbons for energy. The genomic data is used in an artificial intelligence model to identify a drilling site for hydrocarbon production.

### TECHNICAL FIELD

The present disclosure is directed to using genomic analysis to identify specific genomic sequences that can be used to indicate the likelihood that hydrocarbons are present.

### BACKGROUND

The location of crude oil and natural gas reservoirs is performed by a number of geophysical techniques. These may include seismic reflection surveys to image the features of the subsurface environment and identify. In seismic surveys, vibrations that are initiated at the surface travel into the subsurface, and reflect off features, such as rock layers. The reflected vibrations are detected by arrays of seismic detectors at the surface. The signals from the seismic detectors are then processed to generate the images, for example, based on the amount of time it takes the reflected sound waves to travel through different types of rock.

Other techniques are often used in concert with seismic surveys. For example, these can include gravity surveys, magnetic surveys, electromagnetic surveys, and the like. Usually a number of geophysical techniques are used together to generate a likely location for a reservoir that may be used to identify a drilling site. Once a drilling site is identified, survey wells may be used to further refine the information, for example, using drilling logs and analysis of well fluids to determine the type and amount of hydrocarbons present.

In recent years, genomic analyses techniques have progressed to allow the sequencing of the DNA and RNA of diverse organisms. The sequencing techniques allow the identification of different types of bacteria and bacterial communities present in samples, such as soil. These identifications are being explored to determine if they can provide further information for the location of oil and gas reservoirs. For example, the natural seepage of hydrocarbons to surface locations may increase the number of bacterial communities that utilize these hydrocarbons for energy. Accordingly, the presence of bacteria that are known to use hydrocarbons may be an additional piece of information that can be added to other geophysical techniques to identify potential sites for reservoirs.

### SUMMARY

An exemplary embodiment described herein provides a method for using genomic data to locate a reservoir. The method includes collecting samples in a field over a reservoir. A genomic analysis is performed on the samples to obtain genomic data. The genomic data is clustered to classify sequences of microbial communities associated with using hydrocarbons for energy. The genomic data is used in an artificial intelligence model to identify a drilling site for hydrocarbon production.

### BRIEF DESCRIPTION OF DRAWINGS

FIG. 1 is a process flow diagram of a method for using genomic data to enhance the identification of sites for drilling.

FIG. 2 is a schematic drawing of a wellbore that is drilled into a reservoir layer.

FIG. 3 is a top view of the schematic drawing of FIG. 2, illustrating concentration lines for hydrocarbons in the reservoir layer.

FIG. 4 is a process flow diagram of a method for the genomic analysis of samples from the surface or from the near surface layers above a suspected reservoir layer.

FIG. 5 is a drawing showing the clustering of samples based on the similarities of different genomic sequences.

FIG. 6 is a drawing of a multilayer perceptron (MLP) that can be used to predict the locations of hydrocarbons for drilling wells based on genomic data.

FIG. 7 is a view of the schematic drawing of FIG. 2, illustrating isoprobability lines for hydrocarbons based on the output from the MLP of FIG. 6.

FIG. 8 is a block diagram of a computational system that can implement a method for locating a reservoir based on genomic data.

### DETAILED DESCRIPTION

Research has been performed on locating oil reservoirs based on bacterial communities that indicate the presence of crude oil or natural gas. While the identification of bacteria has been studied for enhancing the exploration for hydrocarbons, many communities of bacteria overlap in location, and may not be a strong indicator of the presence of hydrocarbons.

Further, the oil industry lacks a comprehensive description of the organisms in surface locations, near-surface locations, and downhole in hydrocarbon reservoirs. The diversity of microorganisms on or near the surface in hydrocarbon rich fields can be a great source of information about the microbial community genes originating from the hydrocarbon-rich fields. These genes may indicate the presence of different organisms, as well as identifying organisms that can use hydrocarbons for energy in addition to other sources of energy. In addition, correlating surface microorganisms with sub-surface microorganisms from cuttings can provide additional information.

The genomic information may be used to develop a computational tool based on artificial intelligence (AI) algorithms that use the taxonomic and functional microbial information to identify successful hydrocarbon bearing sites. This information can be combined with other geophysical data to enhance the accuracy of locating the hydrocarbon bearing sites, lowering the costs of finding the sites.

In the techniques described herein, biomarkers are developed for oil exploration from the surface by exploring the composition of the bacterial communities in surface soil samples collected from the potential drilling sites. This may be performed by using genomic analysis to determine 16S rRNA sequences, a shotgun metagenomic analysis, or both. As used herein, rRNA is ribosomal ribonucleic acid, which is the primary component of the ribosomes that carry out protein synthesis in a cell. The analysis of the rRNA allows the taxonomic identification of microorganism, such as bacterial communities present in a sample.

As used herein, shotgun metagenomics analyzes samples for genomic material from thousands of organisms in parallel. This approach provides insight into community biodiversity and functions. Further, shotgun sequencing allows for the detection of low abundance members of microbial communities.

Shotgun metagenomics provides genomic data for numerous sequences found in a sample. These sequences can be used to predict proteins that are being generated by the organisms present. For example, an organism that can use hydrocarbons for energy, if present, or other material for energy if hydrocarbons are not present, would express certain sequences if hydrocarbons were present. Further, similar sequences may be present in other types of organisms that use hydrocarbons for energy, allowing the determination of the presence of hydrocarbons without requiring the identification of a specific organism. As used herein, the sequences collected are mathematically represented, for example, by numbers representing the sequence. The sequences constitute genomic data on the microorganisms present in a sample and their metabolic functions.

The genomic data may then be correlated with the genomic material in samples collected from the reservoir. This information may be used in a computational tool based on artificial intelligence (AI) algorithms that identifies successful drilling sites. Further, the whole metagenome shotgun sequencing approaches that investigates the functions of the microorganisms in the fields may improve and generalize the AI-based screening approach.

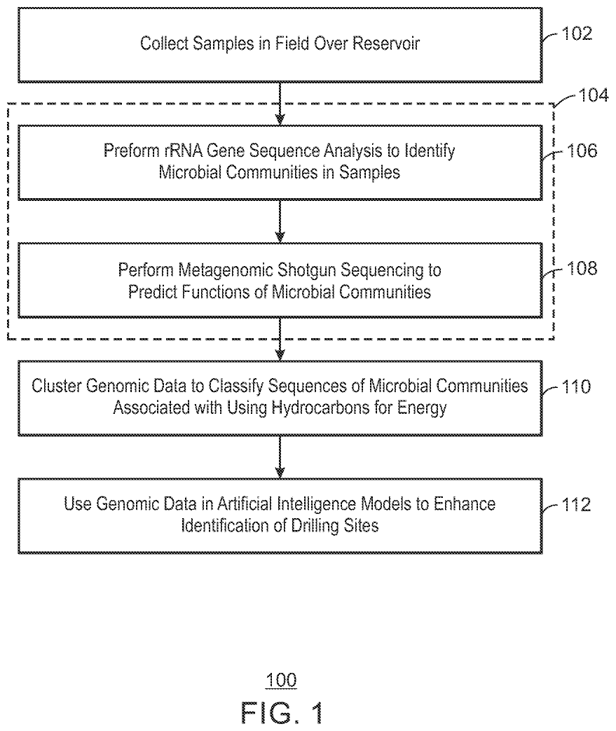

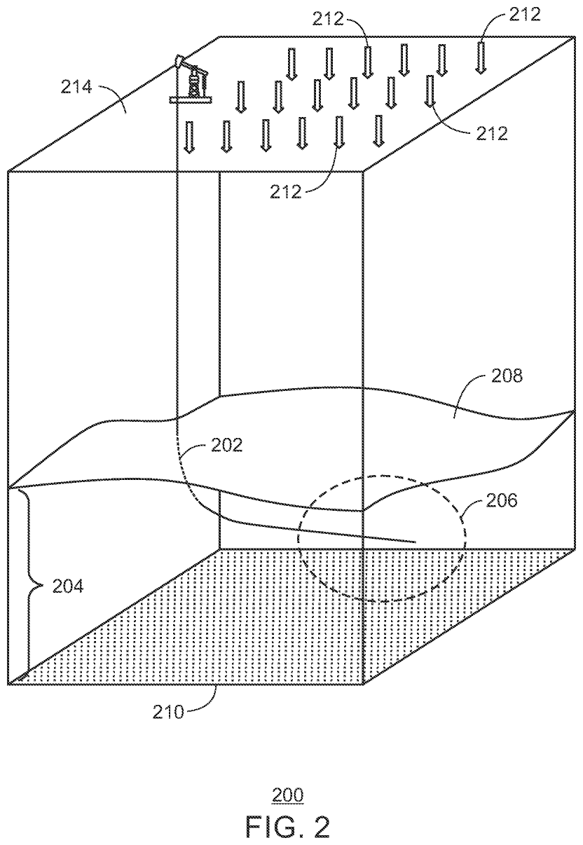

FIG. 1 is a process flow diagram of a method 100 for using genomic data to enhance the identification of sites for drilling. The method begins at block 102 with the collection of samples in an oilfield over a reservoir. The samples may include soil, water, and oil collected from the oilfield. This is discussed further with respect to FIG. 2.

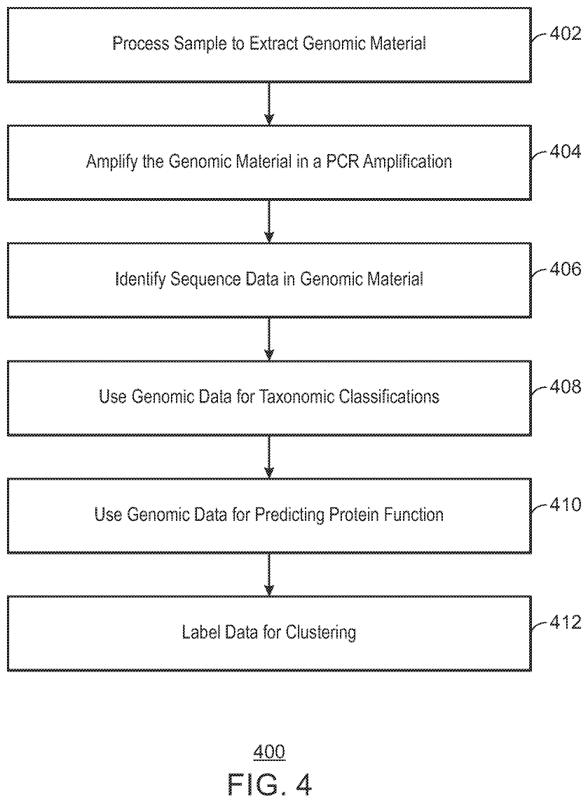

At block 104, a genomic analysis is performed on the samples to obtain genetic data. At block 106, 16S rRNA gene sequencing analysis is performed to identify microbial communities in the samples. This is discussed further with respect to FIG. 4. At block 108, whole metagenome shotgun sequencing analysis is performed to determine the functions of the microbial communities, such as the metabolic potential of the microbial communities to utilize hydrocarbons for energy.

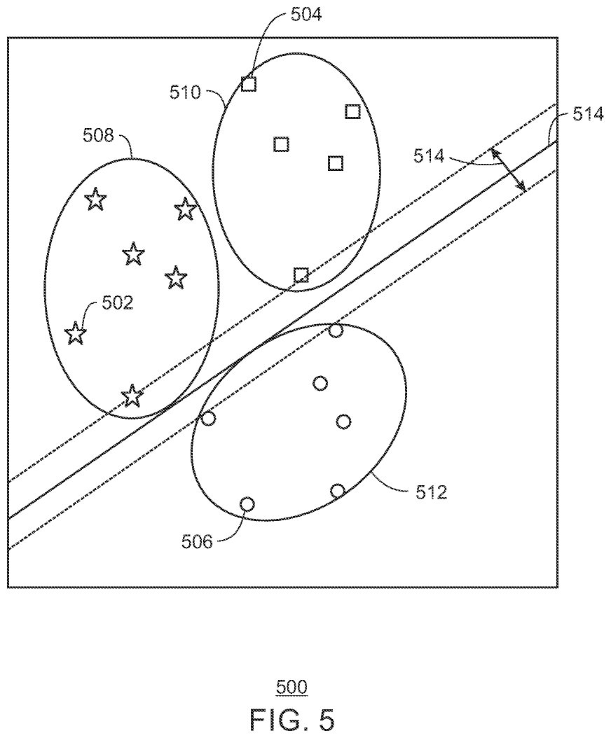

At block 110, the genomic data is clustered to identify functions of microbial communities that are associated with using hydrocarbons for energy. This is discussed further with respect to FIG. 5.

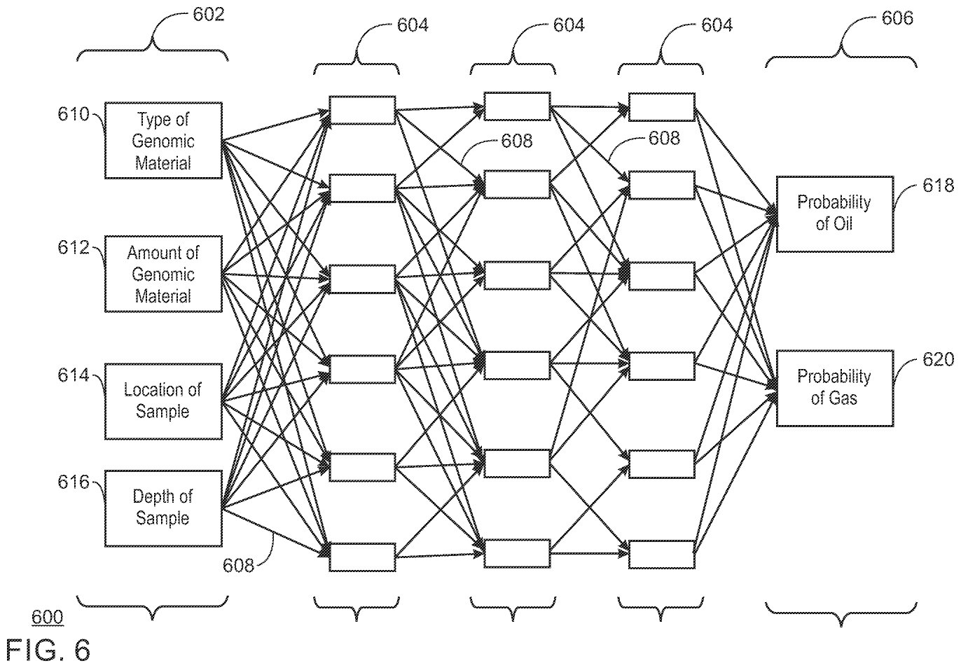

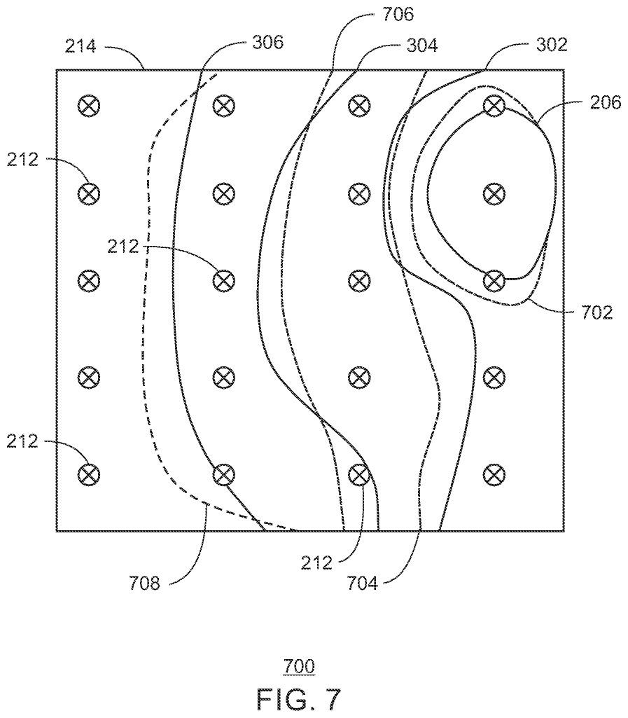

At block 112, the genomic data is used in artificial intelligence models to enhance identification of drilling sites. This is discussed further with respect to FIGS. 6 and 7.

Thus, the techniques provide an approach to use the characterization of the microbial community proximate to oil and gas reservoirs as an assessment criterion for exploration. The techniques provide a number of advantages in addition to identifying biomarkers that may be used for oil exploration. For example, the techniques may be utilized in genetic engineering applications for microbial enhance oil recovery (MEOR). They may also be used to understand the effect of water injection and the quality of reservoir souring. Further, the techniques may be used to determine the potential of microbial oil upgrading, identify effective microbial mitigation techniques, and economically perform a risk assessment of bio-corrosion at the well site.

FIG. 2 is a schematic drawing 200 of a wellbore 202 that is drilled into a reservoir layer 204. Under ideal conditions, the wellbore 202 is placed in a zone 206 that has a maximum concentration of hydrocarbons, for example, located between a cap rock layer 208 and a water layer 210. However, as discussed herein, the challenge in geophysical surveys of reservoirs is to identify the zone 206.

As described herein, samples are collected at sample points 212 along the surface 214, over the anticipated location of the zone 206. To simplify the drawing, not every sample point 212 is labeled. The samples may include samples taken at the surface 214, and samples taken at near surface depths, such as 1 meter (m), 5 m, and 10 m below the surface 214. The samples may also include water samples taken at the surface or in the subsurface, as well as samples taken from the reservoir layer 204.

The samples may be processed to identify genomic information, such as DNA and rRNA, of bacterial communities to identify the bacterial communities present and the functional genes operative in the bacterial communities. As described herein, the information from the locations of the samples, the genomic information, and the amounts and identities of hydrocarbons found may be used in a computational tool relying on an artificial intelligence (AI) to identify successful drilling sites using whole metagenome shotgun sequencing.

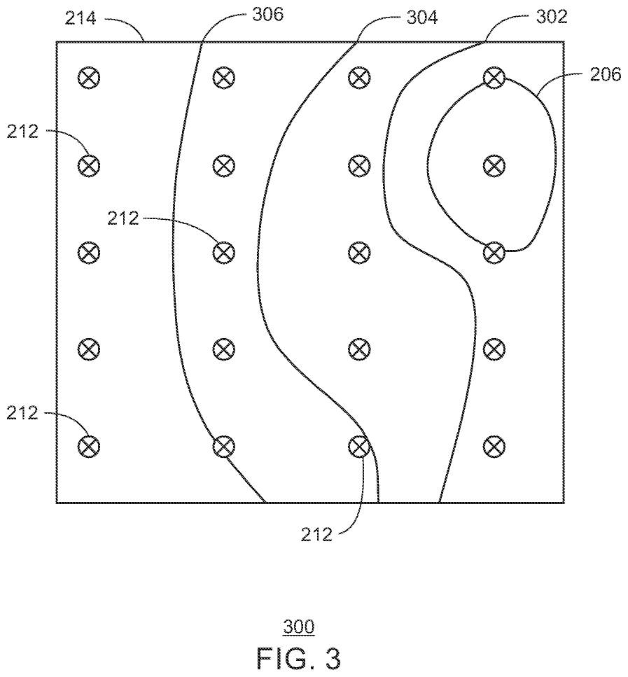

FIG. 3 is a top view 300 of the schematic drawing 200 of FIG. 2, illustrating concentration lines for hydrocarbons in the reservoir layer. Like numbered items are as described with respect to FIG. 2. As can be seen in the top view 300, the zone 206 of maximum concentration of hydrocarbons is not isolated from the rest of the reservoir layer 204 (FIG. 1), but may extend out into the reservoir layer 204 with successive reductions in concentration further out, as illustrated by the concentration lines 302, 304, and 306. Accordingly, the top view 300 can be considered as a map of the concentrations of the hydrocarbons in the reservoir layer 204 projected across the surface 214 over the reservoir layer 204. The microbial communities at each of the sample points 212 may reflect the concentration of hydrocarbons, depending on the amount of hydrocarbons that have made it to the surface, for example, through faults. In some examples, only the sample points 212 directly above the zone 206 may show microbial communities acclimatized to using hydrocarbons for energy.

FIG. 4 is a process flow diagram of a method 400 for the genomic analysis of samples from the surface or from the near surface layers above a suspected reservoir layer. The method begins at block 402 when each sample is processed to extract genomic materials, such as DNA, rRNA, or both (separately). In some embodiments, the genomic material may include tRNA (transfer RNA), to track the active genes in the organism. For example, whole shotgun metagenomics sequencing is used in some embodiments.

At block 404, the genomic material is amplified using a polymerase chain reaction (PCR) amplification. The PCR amplification uses for steps. To begin, the genomic material is heated to separate the double-stranded DNA or rRNA chains into two single strands. The separated strands may then be annealed by reacting with short sequences of 20-30 base pairs to aid in the detection of target sequences. The annealed strands are then treated with an enzyme to replicate the strands, for example, for DNA the enzyme is Taq polymerase. Polymerase is a recombinant thermally stable DNA polymerase isolated from the organism Thermus aquaticus, and is commercially available along with the PCR amplification systems.

If the target strands are RNA, such as 16S rRNA, an rRNA amplicon sequencing approach may be used. This approach is based on amplification of small fragments of one or two hypervariable regions of the 16S rRNA gene. The sequences of these fragments are then obtained and compared with reference sequences in curated databases for taxonomic identification.

In various embodiments, a number of commercially available tools can be used for the whole shotgun metagenomic and 16S rRNA sequence analyses. For example, in some embodiments, the metagenomic workflows are managed using the Arvados software platform, which is available on GitHub and is provided by Arvados.org. Arvados allows the efficient storage of data and the generation of reproducible workflows written in Common Workflows Language (CWL).

At block 406, the sequence data is identified and prepared for use. For example, this may include the use of tools to clean up and check the quality of the sequence reads, such as Trim-galore, which is available on GitHub and is provided by Babraham Bioinformatics, or Trimmomatic, which is available on GitHub and is provided by the Usadel Lab at USADELLAB.org. The sequence data can then be assembled using the strategic k-mer extension for scrupulous assemblies (SKESA) software package available on GitHub, which is provided by the National Center for Biotechnology Information (NCBI) at the National Institutes of Health. SKESA is a de novo sequence assembler that can assemble short nucleotide sequences into longer ones without the use of a reference genome.

At block 408, the genomic data is used for taxonomic classifications. In some embodiments, this is performed using the Kraken2 database. The Kraken2 database is available on GitHub and is provided by the Johns Hopkins University. It supports both whole metagenomic and 16S sequence databases.

At block 410, the genomic data is used for predicting protein function. In some embodiments, this may be performed by the DeepGOPlus software package. DeepGOPlus is available on GitHub and is provided by the King Abdullah University of Science and Technology.

At block 412, the genomic data is labeled and organized for further operations, such as clustering, as indicated in FIG. 5. In some embodiments, the labeling is performed by associating particular sequences with sample locations over the surface above a hydrocarbon reservoir. In various embodiments, labeling is performed by associating particular sequences with the taxonomic identification of particular microbial organisms associated with the use of particular types of hydrocarbons as an energy source. For example, a similar set of sequences found in the shotgun metagenomics approach may be associated with the use of light hydrocarbons, such as methane and ethane, as an energy source, even across multiple microbial communities. This may allow the use of these sequences as bio-markers, even without identifying specific organisms. This is discussed further in the following figures.

FIG. 5 is a drawing 500 showing the clustering of samples based on the similarities of different genomic sequences. Data points for related genomic sequences 502, 504, and 506 are indicated by shapes (stars, squares, and circles). The spread in these points may indicate noise in the data or slight differences in the sequences between different organisms. To perform the clustering, unsupervised machine learning is used first, in particular deep clustering methods based on dimensionality reduction and then exploiting similarity measures to identify clusters. Once the data is clustered, it may be labelled, for example, corresponding to the presence of different types of hydrocarbons, for use in AI models, as described with respect to FIG. 6.

As used herein, dimensionality reduction refers to any number of known techniques that transform data from a high-dimensional space into a low-dimensional space. For the genomic sequences 502, 504, and 506, the transforms are performed so that the low-dimensional representation retains some meaningful properties of the original genomic data. Such techniques may include principal component analysis (PCA), among others. PCA performs a linear mapping of the genomic data from a higher dimension to lower dimension while maximizing the variance in the data. PCA is generally performed by an eigenvector analysis in which the eigenvectors for the data points in the data set are calculated, and then the largest eigenvectors are retained, while smaller eigenvectors are discarded. The lower dimension genomic data is then regenerated from the eigenvectors.

After the dimensionality reduction of the genomic sequences 502, 504, and 506 is performed, similarity measures may be used to identify clusters, such as clusters 508, 510, and 512, including, for example, distance measures between data points. This may include grouping points by Euclidean distance calculations between points in multidimensional space, among other distance measures. Once the points are grouped into clusters, various techniques may be used to assist in labeling which clusters of genomic data are related to the presence of hydrocarbons. Other types of clustering may include rotational clustering, density based clustering, or hierarchical clustering among others. Other clustering techniques known in the art may be used.

Labelling of the cluster genomic data may be manually performed, for example, by correlating sequences in particular clusters, for example, clusters 508 and 510, with the ability to use hydrocarbons for energy. The labelling may also be performed by algorithmic techniques, such as support vector machines.

Generally, support-vector machines (SVM) are supervised learning models with associated learning algorithms that analyze data for classification and regression analysis. SVMs are one of the most robust prediction methods, being based on statistical learning frameworks or VC theory. Given a set of training examples, each labeled as belonging to one of two categories, such as sequence associated or not associated with the presence of hydrocarbons, an SVM training algorithm builds a model that assigns new examples to one category or the other, making it a non-probabilistic binary linear classifier. As shown in FIG. 5, an SVM maps training examples, such as the genomic sequences 502, 504, and 506 to points in space to maximize the width of the gap 514 between the two categories. New genomic sequences 502, 504, and 506 are then mapped into that same space and predicted to belong to a category based on which side of the gap they fall.

In some embodiments, SVMs may be used for clustering (unsupervised learning) and labelling the genomic sequences 502, 504, and 510. An SVM-based clustering algorithm that clusters data with no labeling of input classes may be performed. The algorithm first runs a binary SVM classifier against a data set with each genomic sequence 502, 504, or 506 either labelled manually, or randomly labelled. This is repeated until an initial convergence occurs. Once the first runs are complete, the confidence parameters for the classification of each of the genomic sequences 502, 504, and 506 can be accessed. The genomic sequences 502, 504, and 506 with the lowest confidence in the labels have the labels switched to the other class label, for example, associate with the presence of hydrocarbons. The SVM is then run again on the genomic data 502, 504, and 506. The SVM technique improves on the convergence results by rerunning the SVM after relabeling the genomic sequence with the lowest confidence levels, for example, using a threshold value for the confidence levels to determine when to relabel a genomic sequence 502. The labeled and clustered genomic sequences can then be used in AI models.

FIG. 6 is a drawing of a multilayer perceptron (MLP) 600 that can be used to predict the locations of hydrocarbons for drilling wells based on genomic data. An MLP 600 is a type of neural network model. Generally, as shown in FIG. 6, the MLP 600 consists of at least three layers of nodes: an input layer 602, one or more hidden layers 604, and an output layer 606. Except for the input layer 602 of node, each node is a neuron that uses a nonlinear activation function based on weighted hyperparameters 608. In the example shown in FIG. 6, the input layer 602 is coupled to the output layer 606 by three hidden layers 604 of nodes.

In the example shown in FIG. 6, the input layer 602 includes nodes for the type of genomic material 610, the amount of genomic material 612, the location 614 the genomic sample was collected, and the depth 616 the sample was collected, such as 1 m, 5 m, or 10 m. Depending on the data, desired outputs, and numerical versus binary outputs, the input data may be adjusted to use fewer or more inputs. The outputs in this example are the probability of oil 618, or heavy hydrocarbons, being present at a location 614, and the probability of gas 620, or light hydrocarbons, being present at the location 614.

The MLP 600 utilizes a supervised learning technique called backpropagation for training. In this technique, a training set of values are placed at the input layer 602, and an error function is calculated for the values at the output layer 606. The hyperparameters 608 are then tuned until the error at the output layer 606 is within an acceptable tolerance limits, for example, 1%, 5%, or 10%, or higher. The AI model can then be used with new values to build a map of the probable locations of the hydrocarbons, as described with respect to FIG. 7.

FIG. 7 is a view 700 of the schematic drawing of FIG. 2, illustrating isoprobability lines 702-708 for hydrocarbons based on the output from the MLP 600 of FIG. 6. Like numbered items are as described with respect to FIG. 2. Once the MLP 600 is trained, it can be used on a new data set, for example, following the method 100 of FIG. 1. The output of the AI model may be used to provide isoprobability lines 702-708 based on the genomic sequences in the samples taken from each of the sample points 212. As shown in the view 700 of FIG. 7, one of isoprobability lines 702 indicates a zone of highest probability of finding hydrocarbons, and may correlate with the concentration lines 302, 304, and 306 corresponding to the actual concentrations of hydrocarbons in the reservoir layer 204.

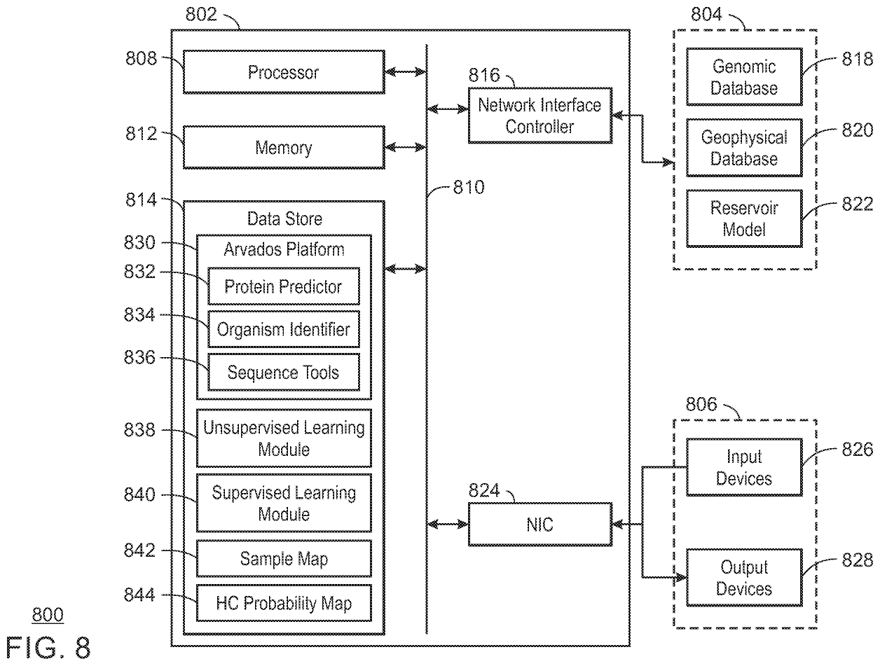

FIG. 8 is a block diagram of a computational system 800 that can implement a method for locating a reservoir based on genomic data. The computational system 800 includes a computing unit 802, an external network 804, and I/O devices 806. In some embodiments, the computing unit 802 is a computer, a workstation, or a laptop, among others. In other embodiments, the computing unit 802 is a virtual machine running on a processor in a cloud computing system, on a virtual processor in a cloud server, or using other real or virtual processors.

The computing unit 802 includes a processor 808. The processor 808 may be a microprocessor, a multi-core processor, a multithreaded processor, an ultra-low-voltage processor, an embedded processor, or a virtual processor. In some embodiments, the processor 808 may be part of a system-on-a-chip (SoC) in which the processor 808 and the other components of the computing unit 802 are formed into a single integrated electronics package. In various embodiments, the processor 808 may include processors from Intel® Corporation of Santa Clara, Calif., from Advanced Micro Devices, Inc. (AMD) of Sunnyvale, Calif., or from ARM Holdings, LTD., Of Cambridge, England. Any number of other processors from other suppliers may also be used.

The processor 808 may communicate with other components of the computing unit 802 over a bus 810. The bus 810 may include any number of technologies, such as industry standard architecture (ISA), extended ISA (EISA), peripheral component interconnect (PCI), peripheral component interconnect extended (PCIx), PCI express (PCIe), or any number of other technologies. The bus 810 may be a proprietary bus, for example, used in an SoC based system. Other bus technologies may be used, in addition to, or instead of, the technologies above.

The bus 810 may couple the processor 808 to a memory 812. The memory 812 include any number of volatile and nonvolatile memory devices, such as volatile random-access memory (RAM), static random-access memory (SRAM), flash memory, and the like. The memory 812 holds currently operating programs, systems, and results.

The bus 810 may couple the processor 808 to a data store 814. The data store 814 is used for the persistent storage of information, such as data, applications, operating systems, and so forth. The data store 814 may be a nonvolatile RAM, a solid-state disk drive, or a flash drive, among others. In some embodiments, the data store 814 will include a hard disk drive, such as a micro hard disk drive, a regular hard disk drive, or an array of hard disk drives, for example, associated with a network or cloud server.

The bus 810 couples the processor 808 to a network interface controller 816. In some embodiments, the network interface controller 816 connects the computing unit 802 to data sources and sinks located in the external network 804, for example, through an Ethernet connection. The external network 804 may be a local network, a corporate intranet, or the Internet, among others. In various embodiments, the data sources and sinks include a genomic database 818 that provides taxonomic information, sequence information, or both. The genomic database 818 may include information provided by outside sources, such as academic and private research organizations, as well as information provided by the techniques described herein.

A geophysical database 820, for example, for a particular field, may provide seismic images and other geophysical data to be used along with the genomic information from the present techniques in a reservoir model 822. The reservoir model 822 may use the assembled data to identify sites for drilling.

The bus 810 couples the processor 808 to a human machine interface (HMI) 824. The HMI 824 couples the computing unit 802 to the I/O devices 806. The I/O devices 806 include input devices 826, such as keyboards, pointing devices, and microphones, among others. The I/O devices 806 include output devices 828, such as monitors, printers, plotters, and speakers, among others.

The data store 814 includes blocks of stored instructions that, when executed, direct the processor 808 to implement the functions of the computational system 800. The data store 814 includes a block 830 of instructions that operates a genomic computing platform, such as the Arvados computing platform, or a similar computing platform. As described herein, the instructions in block 830 may host a number of applications, such as a block 832 of instructions that predict organism functions from genomic sequences, such as a protein predictor. Another block 834 of instructions may perform taxonomic identifications from genomic sequences, such as 16S rRNA sequences. In various embodiments, the genomic computing platform hosts one or more blocks 836 of instructions that perform sequence operations, such as cleaning and verification.

The data store 814 includes a block 838 of instructions that implements an unsupervised learning module. As described herein, the unsupervised learning module may use techniques for dimensional reduction, such as principal component analysis, to decrease the dimensions in the data prior to clustering the data. The clustering may be performed by distance measurements, unsupervised SVMs, and the like.

The data store 814 may include a block 840 of instructions that implements a supervised learning technique for identifying highest probability regions for oil drilling. The supervised learning techniques may include neural networks, supervised training SVMs, and the like.

The data store 814 may also include data on the analysis, such as a sample map 842, mapping the genomic data-to-data collection locations and depths. A predicted hydrocarbon probability map 844 may store the hydrocarbon probabilities for each location, as determined by the model implemented by the supervised learning module.

### EMBODIMENTS

An exemplary embodiment described herein provides a method for using genomic data to locate a reservoir. The method includes collecting samples in a field over the reservoir. A genomic analysis is performed on the samples to obtain genomic data. The genomic data is clustered to classify sequences of microbial communities associated with using hydrocarbons for energy. The genomic data is used in an artificial intelligence model to identify a drilling site for hydrocarbon production.

In an aspect, the method includes collecting the samples in a grid over a surface of the field. In an aspect, the method includes collecting the samples in subsurface layers of the field. In an aspect, the method includes collecting the samples from the reservoir. In an aspect, the method includes collecting samples from cuttings obtained during drilling.

In an aspect, the method includes extracting genomic material from the samples. In an aspect, the method includes amplifying the genomic sequences in a PCR amplification process. In an aspect, the method includes identifying the sequences present in the genomic material.

In an aspect, the method includes performing rRNA gene sequence analysis to identify the microbial communities in the samples. In an aspect, the method includes associating the identity of the microbial communities with hydrocarbons.

In an aspect, the method includes performing a whole shotgun metagenomic sequencing to obtain the genomic data. In an aspect, the method includes correlating the genomic data with metabolic functions. In an aspect, the method includes labelling the genomic data of microbial communities associated with using hydrocarbons for energy.

In an aspect, the method includes performing a dimensionality reduction on the genomic data. In an aspect, the method includes performing the dimensionality reduction using a principal component analysis.

In an aspect, the method includes clustering the genomic data through Euclidian distance calculations. In an aspect, the method includes clustering the genomic data through an unsupervised learning support vector machine.

In an aspect, the method includes constructing a multilayer perceptron coupling to identify drilling sites. In an aspect, the method includes constructing the multilayer perceptron to use genomic data as an input and probability of hydrocarbons as an output. In an aspect, the method includes training the multilayer perceptron by adjusting weights of hyperparameters between nodes.

Other implementations are also within the scope of the following claims.

What is claimed is:

1. A method for using genomic data to locate a reservoir, comprising:
collecting samples in a field over the reservoir;
performing genomic analysis on the samples to obtain genomic data;
clustering the genomic data to classify sequences of microbial communities associated with using hydrocarbons for energy; and
using the genomic data in an artificial intelligence model to identify a drilling site for hydrocarbon production.

2. The method of claim 1, comprising collecting the samples in a grid over a surface of the field.

3. The method of claim 1, comprising collecting the samples in subsurface layers of the field.

4. The method of claim 1, comprising collecting the samples from the reservoir.

5. The method of claim 1, comprising collecting samples from cuttings obtained during drilling.

6. The method of claim 1, comprising extracting genomic material from the samples.

7. The method of claim 6, comprising identifying the genomic sequences present in the genomic material.

8. The method of claim 7, comprising amplifying the genomic sequences in a PCR amplification process.

9. The method of claim 1, comprising performing rRNA gene sequence analysis to identify the microbial communities in the samples.

10. The method of claim 9, comprising associating the identity of the microbial communities with hydrocarbons.

11. The method of claim 1, comprising performing a whole shotgun metagenomic sequencing to obtain the genomic sequence.

12. The method of claim 11, comprising correlating the genomic data with metabolic functions.

13. The method of claim 12, comprising labelling the genomic data of microbial communities associated with using hydrocarbons for energy.

14. The method of claim 1, comprising performing a dimensionality reduction on the genomic data.

15. The method of claim 14, comprising performing the dimensionality reduction using a principal component analysis.

16. The method of claim 1, comprising clustering the genomic data through Euclidian distance calculations.

17. The method of claim 1, comprising clustering the genomic data through an unsupervised learning support vector machine.

18. The method of claim 1, comprising constructing a multilayer perceptron coupling to identify drilling sites.

19. The method of claim 18, comprising constructing the multilayer perceptron to use genomic data as an input and probability of hydrocarbons as an output.

20. The method of claim 18, comprising training the multilayer perceptron by adjusting weights of hyperparameters between nodes.
